(19) United States
(12) Patent Application Publication
(10) Pub. No.: US 2017/0073548 A1
(43) Pub. Date: Mar. 16, 2017

(54) CONFORMING SECUREMENT ARTICLES AND METHODS OF USE

(71) Applicant: 3M INNOVATIVE PROPERTIES COMPANY, St. Paul, MN (US)

(72) Inventors: Naimul Karim, Maplewood, MN (US); Michael G. Williams, Vadnais Heights, MN (US)

(21) Appl. No.: 15/363,412

(22) Filed: Nov. 29, 2016

Related U.S. Application Data
(62) Division of application No. 14/395,699, filed on Oct. 20, 2014.
(60) Provisional application No. 61/637,013, filed on Apr. 23, 2012.

(51) Int. Cl. C09J 7/02; A61L 15/58; A61M 25/02; B32B 5/02; B32B 27/00; B32B 37/12; B32B 5/18; C09J 5/00; B32B 1/08

(52) U.S. Cl. CPC C09J 7/02; C09J 5/00; A61L 15/58; A61M 25/02; B32B 1/08; B32B 27/00; B32B 37/12; B32B 5/18; B32B 5/024; C09J 2201/16; A61M 2025/0266; A61M 2207/00; B32B 2305/022; B32B 2305/188; B32B 2405/00; B32B 2535/00

(57) ABSTRACT

An article for securing an object to a surface is provided. The article comprises a conformable backing having top and bottom major surfaces and a perimeter edge, an adhesive coated on at least a portion of the bottom major surface of the backing, and a self-supporting carrier releasably adhered to the top major surface of the backing, the carrier having spaced-apart first and second sections, each section having a central edge. The central edges define opposing borders of an object-conformable region of the backing. On opposite sides of the object-conformable region, the backing comprises two anchor regions. Also provided is a method of using the article to secure an object to the surface. The method comprises using at least one section of the carrier to conform the backing to at least a portion of the topological shape of the object to be secured to the surface.

### CROSS REFERENCE TO RELATED APPLICATIONS

This application is a divisional application of U.S. patent application Ser. No. 14/395,699, filed Oct. 20, 2014, which is a 371 of International PCT/US2013/026621, filed Feb. 19, 2013, which claims the benefit of U.S. Provisional Patent Application No. 61/637,013, filed Apr. 23, 2012, which is incorporated herein by reference in its entirety.

### BACKGROUND

Securing medical devices to a patient using tape, however, has several drawbacks. First, the use of tape to secure a medical device can retain dirt or other contaminants at or near the skin or a break in the skin (e.g., catheter insertion site), potentially leading to infection or other complications. Indeed, numerous clinical studies have implicated improperly secured catheters in a wide range of complications including, for example, catheter-related blood stream infections (CRBSI). Second, tape often fails to limit movement of the medical device in one or more directions, and thus can contribute to motion-related complications such as bruising, phlebitis, extravasation, infiltration, and catheter migration, which can lead to catheter dislodgement or disconnection. Third, tape removal can itself cause undesired movement of the medical device. Fourth, tape must periodically be changed, often daily. The frequent removal and reapplication of adhesive tape can irritate a patient's skin, as well as lead to the buildup of adhesive residue on the outer surface of the catheter (or other medical device). Such adhesive residue not only makes the catheter (or other medical device) stickier and more difficult for healthcare providers to handle, it can also result in contaminants (including pathogens) adhering to the medical device itself, increasing the likelihood of infection, either at the skin surface or internally. Fifth, tape securement can allow medical devices attached to a patient to flex or kink, which can lead to clinical complications.

There is a need for improved articles and methods to secure a medical device to a patient.

### SUMMARY

In general, the present disclosure relates to articles and methods used to secure an object to a surface. The article comprises a backing with an adhesive layer disposed thereon. The article further comprises a carrier that is configured to help the operator apply the securement article to the object and to the surface to which the object is to be secured. The carrier comprises at least two sections that are releasably adhered to the backing on the side opposite the adhesive layer. Advantageously, the carrier sections are used to guide the backing during the process of securing the article and, subsequently, one or more of the carrier sections can be removed and discarded.

In one aspect, the present disclosure provides an article. The article can comprise a conformable backing having top and bottom major surfaces and a perimeter edge, an adhesive coated on at least a portion of the bottom major surface of the backing, and a self-supporting carrier releasably adhered to the top major surface of the backing, the carrier having spaced-apart first and second sections, each section having a central edge, wherein the central edges define opposing borders of an object-conformable region of the backing.

In any embodiment, wherein the backing further can comprise two conformable anchor regions located on opposite sides of the object-conformable region. In any of the above embodiments, the backing can comprise a sheet material comprising polymer film, a foam, a woven fabric, a nonwoven fabric, or a combination of any two or more of the foregoing sheet materials. In any of the above embodiments, the sheet material can comprise an elastic sheet material. In any of the above embodiments, the complementary edges can be complementary shaped or non-complementary shaped.

In any of the above embodiments, the object-conformable region can be substantially transparent. In any of the above embodiments, the adhesive can comprise an iso-octyl acrylate:acrylamide copolymer, an isooctyl acrylate:ethyleneoxide acrylate:acrylic acid terpolymer, a derivative of either of the foregoing adhesives, a silicone adhesive, or a mixture of any two or more of the foregoing adhesives. In any of the above embodiments, the adhesive can be pattern-coated. In any embodiment, the article further can comprise a liner releasably adhered to the adhesive.

In any of the above embodiments, the carrier can comprise a material selected from the group consisting of self-supporting heavy-duty paper, cardstock, cardboard, and self-supporting polymeric sheet materials, and a mixture or a combination of any two or more of the foregoing materials. In any of the above embodiments, the carrier can comprise a material that is optically-transmissive. In any of the above embodiments, the object-conformable region can comprise a first midline, wherein the first midline can define a substantially straight line, a curved line, or an angular line. In any of the above embodiments, the article further can comprise a third section of the carrier releasably adhered to the top face of the object-conformable region of the backing. In any of the above embodiments, the object-conformable region further comprises at least one slit located along the perimeter of the backing at the object-conformable region. In any of the above embodiments, the carrier further can comprise a tab. In any of the above embodiments, the article further can comprise an adhesive strip releasably adhered to the top face of the carrier. In any of the above embodiments, the article further can comprise a reinforcement layer.

In another aspect, the present disclosure provides a method. The method can comprise providing an object having a topological shape, an article according to any one of the above embodiments, and a surface on which to secure the device; positioning the device and article proximate the surface; using at least one section of the carrier to conform the backing to at least a portion of the topological shape; and securing the backing to the surface. In any embodiment of the method, securing the backing to the surface can comprise using the central edge of at least one section of the carrier to cause contact between the adhesive and a portion of the surface proximate the device. In any embodiment of the method, using at least one section of the carrier can comprise using both the first and second sections of the carrier. In any embodiment of the method, using the first and second sections of the carrier further comprises sequentially or simultaneously using the first and second sections.

In any of the above embodiments of the method, using the central edge of at least one section can comprise using the central edges of the first and second sections of the carrier. In any of the above embodiments of the method, using the central edge can comprise using the central edge to reduce tenting between the backing and the surface. In some embodiments of the method, using the central edges can comprise using the edges to provide contact between the article and the device substantially completely around a circumference of the device.

In any of the above embodiments of the method, the object can comprise a medical device. In some embodiments, the medical device is selected from the group consisting of a tube, a catheter, or an electrode lead.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, an edge can be interpreted to mean “one or more” edges.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF THE DRAWINGS

FIG. 1a-d are side views of intermediate steps in a prior art process for securing a medical device to a surface using adhesive tape.

FIG. 2a is a top perspective exploded view of one embodiment of a conforming securement article having a carrier comprising spaced-apart first and second sections according to the present disclosure.

FIG. 2b is a side view of the article of FIG. 2a.

FIG. 2c is a plan view of the article of FIG. 2a.

FIGS. 3a-d are side views of intermediate steps in a process for securing an object to a surface using a conforming securement article according to the present disclosure.

FIG. 4a is a plan view, partially in section, of one embodiment of a conforming securement article having a carrier comprising first, second, and third carrier sections according to the present disclosure.

FIG. 4b is a side view of the article of FIG. 4a.

FIG. 5a is a plan view of one embodiment of a conforming securement article having a carrier comprising first, second, third, and fourth carrier sections according to the present disclosure.

FIG. 5b is a side view of the article of FIG. 5a.

FIGS. 6a-f are side views of intermediate steps in a process for securing a medical device to a surface using the article of FIGS. 5a-b.

FIGS. 7a-e are plan views of five alternative embodiments illustrating different shapes of a conformable securement article according to the present disclosure.

FIGS. 8a-d are plan views of four alternative embodiments of a conformable securement article having a slit according to the present disclosure.

FIG. 9 is a plan view of one embodiment of a conformable securement article adapted to secure a curvilinear medical device according to the present disclosure.

FIG. 10 is a plan view of one embodiment of a conformable securement article adapted to secure an angular medical device according to the present disclosure.

FIGS. 11a-f are side views of intermediate steps in one embodiment in a process for securing an object to a surface, such that at least a part of the object is held off the surface, using a conforming securement article according to the present disclosure.

FIG. 12a is one embodiment of a conformable securement article having an object-conformable region defined by one carrier section having a curved central edge and another carrier section having a substantially linear central edge, according to the present disclosure.

FIG. 12b is one embodiment of a conformable securement article having an object-conformable region defined by one carrier section having an angular central edge and another carrier section having a substantially linear central edge, according to the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to conformable articles that can be used to secure an object to a surface. The articles include a backing that is capable of conforming to the topological shape of the object to be secured. The backing includes an adhesive disposed on at least one major surface. The adhesive (e.g., a pressure-sensitive adhesive) enables the backing to adhere to a surface. Thus, the inventive articles can be used to secure a variety of objects. In addition, the backing is also capable of conforming to the topological shape of a variety of surfaces (e.g., smooth surfaces, rough surfaces, curved surfaces, uneven surfaces). The articles also include a removable carrier that is used to position the backing in close proximity to the object when securing the object to a surface. Positioning the backing in close proximity to the surface provides several advantages including, for example, reducing or precluding the accumulation of undesirable material or contamination between the object and the backing, providing maximal adhesion of the backing (e.g., by maximizing the contact surface between the object and the adhesive on the backing) to the object and/or the surface to which the object is secured, reducing the profile of the secured object, thereby reducing the probability it will be disturbed by other objects that are moving in close proximity to the secured object.

For at least the reasons discussed above, articles of the present disclosure are particularly well-suited for securing medical devices to a variety of surfaces (e.g., a patient's skin, a table, a bedrail). The multi-part carrier of the inventive article facilitates rapid and proper application of the securement article to the object to be secured and the surface to which the object is to be secured. In a preferred embodiment in which the carrier comprises carrier tabs, the tabs can be used to prevent contact between the operator's hands (or gloves) and the adhesive disposed on the backing. In addition, the tabs can be used to separate the carrier sections from the backing after the backing has been applied to a surface.

The following description and the accompanying drawings, which describe and show the certain embodiments, demonstrate several possible representative configurations that an object securement article can take according to the present disclosure. The illustrated embodiments are shown in use with an illustrative example of a urinary catheter. These illustrations are not intended to limit the invention to the specified embodiments or usage. Those skilled in the art will recognize that the described aspects and features of the invention are not limited to any particular embodiment of a securement article, and securement articles according to the invention can readily be designed for use with a variety of objects including, for example, catheters (e.g., urinary catheter, intravenous catheter), catheter hubs, tubes (e.g., enteral feeding tube, chest tube, nasogastric tube,) and electrode leads.

The present disclosure provides an object securement article for securing a medical device to a patient. The medical device preferably has an elongated body that is engaged by the securement article to arrest movement of the medical device in longitudinal, lateral, and other directions when the medical device is secured by the article.

This present disclosure concerns an article and method for securing objects (e.g., medical lines, needles, catheters, etc.) to a surface (e.g., a patient's skin). Such articles include a conformable backing (e.g., a polymer film). The securement article can be highly conformable and, thus, can be adapted to fit a wide range of medical article types, combinations, and dimensions for easy, rapid, and secure attachment to a patient's skin. Because the carrier that is used to apply the article is removable, the portion of the article that contacts the skin also conforms to the skin, forming a smooth, low-profile surface that is less likely to cause patient discomfort and/or become undesirably engaged with clothing or a foreign object, which could result in unexpected detachment of the article and/or medical device.

In each of the embodiments described herein, the conforming securement article has a conformable (e.g., flexible), optionally planar backing that is deformed to enclose a three-dimensional object. The removable carrier sections of the article can be used to position the backing properly during use.

The conforming securement article of each embodiment described herein includes an object-conformable region flanked by two anchor regions disposed on opposite sides of the object-conformable region. When used to secure objects to a patient, the adhesive should be suitable for securing the article to skin and suitable for prolonged skin contact (e.g., for more than one hour, up to and including 1, 3, 5, or more days).

In any embodiment, the articles may be substantially planar when packaged, allowing for easy, dense packaging. Optionally, each article may be individually packaged and/or sterilized or decontaminated, using sterilization or decontamination processes known in the art, before or after packaging. To facilitate a complete understanding, the remainder of the detailed description describes conforming securement articles by reference to the drawings, wherein like elements among the embodiments are referenced with like numerals throughout the following description.

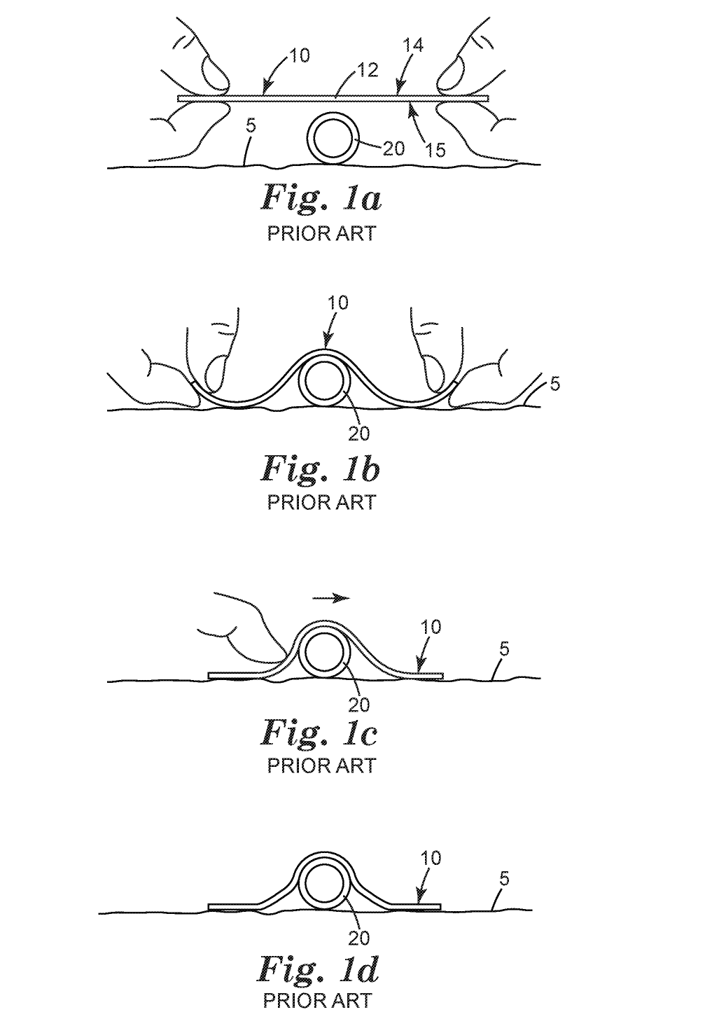

To illustrate a problem that is encountered when securing an object (e.g., a medical device) to a surface, FIG. 1a-d shows side views of a series of intermediate steps in a prior art process used to secure a medical device to a surface using a prior art secural article (e.g., a piece of adhesive tape). The tape 10 comprises a flexible substrate 12 having first and second major surfaces (14 and 15, respectively). One of the major surfaces (surface 15) of the substrate 12 has an adhesive layer (not shown) coated thereon. Initially, when securing the device 20 (e.g., a urinary catheter) to the surface 5 (e.g., a skin surface), the medical device 20 is positioned proximate the surface 5 and the tape 10 is grasped with both hands at opposite ends, positioned over the surface 5 and medical device 20 with the adhesive layer facing the device 20 and the surface 5 (as shown in FIG. 1a), and the ends are moved toward the surface 5 to contact the tape 10 with the device 20 and the surface 5 (as shown in FIG. 1b). Subsequently, fingertip pressure is applied along the nonadhesive side (major surface 14) of the tape 10 to facilitate adhesion of the tape 10 to the medical device 20 and surface 5 (as shown in FIG. 1c). Either through careless application of the tape 10 or by the movement of the device 20 relative to the surface 5 after the device is secured to the surface (e.g., by incidentally contacting the secured device with a hand or other object), “tenting” (i.e., the formation of substantial air gaps 70) can occur between the tape 10 and the medical device 20 that is secured to the surface 5.

The conforming securement articles of the present disclosure include a flexible substrate (i.e., a substrate that can readily conform to the exterior shape of a medical article to be retained) and a unique carrier that facilitates the process of contacting the article with the medical device and the substrate in a way that substantially reduces the incidence of tenting, both during and after the process of securing the object (e.g., medical device) to the surface, when compared to prior art methods.

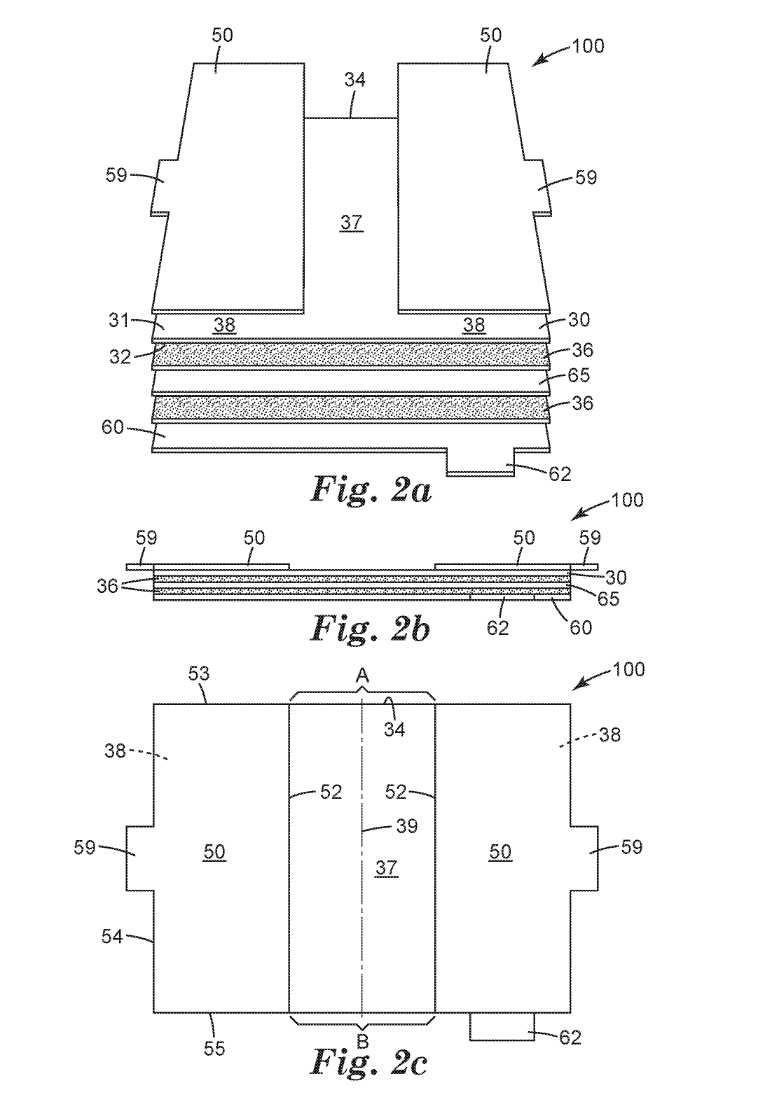

FIGS. 2a-c show several views of one embodiment of a conforming securement article 100 according to the present disclosure. The article 100 comprises a conformable backing 30. “Conformable”, as used herein, refers to the capability of the backing to deform such that one portion of the backing can adapt to enclose the topological shape of an object to be secured while simultaneously another portion of the backing can adapt, if necessary, to the topological shape of the surface to which the object is to be secured. The backing 30 has first (“top”) and second (“bottom”) major surfaces (31 and 32, respectively) and a perimeter edge 34 that defines the area of the backing. Disposed (e.g., as a layer) on at least a portion or the entire area of the second major surface 32 is an adhesive (e.g., adhesive layer 36).

Suitable materials for the backing 30 include deformable sheet materials such as, for example, film (e.g., polymer films) sheets, foam sheets, woven fabric sheets, nonwoven fabric sheets, and combinations of any two or more of the foregoing sheet materials. In some embodiments, the backing 30 may be a highly flexible polymer film. In any embodiment, the backing 30 may have elastic properties (e.g., the backing may comprise an elastic film, foam, or woven, or nonwoven material). Nonlimiting examples of suitable polymer films (e.g., elastomeric polyurethane, polyester, or polyether block amide films) are disclosed in U.S. Pat. Nos. 5,531,855; 6,169,224; 5,738,642; and 6,685,682, the disclosures of which are incorporated herein by reference in their entirety.

The article 100 optionally can comprise a conformable reinforcement layer 65. The reinforcement layer 65 is adhered to the second major surface 32 by adhesive layer 36. In some embodiments, the reinforcement layer 65 may be a film/adhesive laminate, such as HYTREL (DuPont, Wilmington, Del.) film and tackified acrylate adhesive such as a copolymer of iso-octyl acrylate, acrylic acid and FORAL 85 (a triglyceryl ester of reduced abietic acid, commercially available from Hercules Chemical Co., Wilmington, Del.) tackifier, as disclosed in U.S. Patent Application Publication No. 2009/0187130, which is incorporated herein by reference in its entirety. In some embodiments, the reinforcement layer 65 may be a fabric/adhesive laminate. Examples of nonwoven fabric/adhesive laminates include embodiments such as disclosed in U.S. Pat. No. 4,366,814 and available commercially as STEM-STRIP,” (3M, St. Paul, Minn.) elastic skin closure, a nonwoven elastomeric melt blown web of thermoplastic elastomeric small diameter fibers, or CEREX (Monsanto, St. Louis, Miss.) spun bonded nylon and adhesive. Woven fabric/adhesive laminates include embodiments such as cotton cloth laminated to a rubber based adhesive. If the reinforcement layer 65 is not a fabric/adhesive laminate, a second adhesive layer 36 (e.g., a pressure-sensitive adhesive) is applied to the surface of the reinforcement layer 65 opposite the backing 30 to enable the reinforcement layer 65 to adhere to a surface. The second adhesive layer 36 may comprise an adhesive as described herein.

The reinforcement layer 65 may extend over the entire area defined by the backing 30 or may cover only a portion (e.g., the portion that includes all or a part of the object-conformable region 37 or a portion that includes all or a part of at least one of the anchor regions 38) of the area defined by the backing 30. In some embodiments, the reinforcement layer 65 may comprise a conformable nonwoven fabric (e.g., spunlace fabric). Suitable reinforcing layers 65 not only permit the backing to conform to the topological shape of the object to be secured and, if necessary, the topological surface of the surface to which the object is to be secured, they also provide structural reinforcement to the entire backing 30 or a portion of the backing 30 (e.g., a portion of the backing proximate the edges (not shown)). In addition, the reinforcement layer 65 may help the article 100 resist strong external forces (e.g., twisting forces) that might tear an otherwise suitable conformable backing 30 material. Preferably, the reinforcement layer 65 is sufficiently optically transmissible to permit observation of the object to be secured through the backing and the reinforcement layer.

In any embodiment, the adhesive may comprise a pressure sensitive adhesive. Preferred pressure sensitive adhesives, which can include adhesive composites, include adhesives that are typically used for skin-contact applications such as, for example, the acrylate copolymers described in U.S. Pat. No. RE 24,906, the disclosure of which is incorporated herein by reference in its entirety, particularly a 97:3 iso-octyl acrylate:acrylamide copolymer. Adhesive composites include, for example, the adhesive composites described in U.S. Pat. No. 5,531,855, the disclosure of which is incorporated herein by reference in its entirety. Also preferred is an 70:15:15 isooctyl acrylate:ethyleneoxide acrylate:acrylic acid terpolymer adhesive composition, as described in U.S. Pat. No. 4,737,410 (Example 31), the disclosure of which is incorporated herein by reference in its entirety. Other useful adhesives are described in U.S. Pat. Nos. 3,389,827; 4,112,213; 4,310,509; and 4,323,557, the disclosures of which are incorporated herein by reference in their entirety. Inclusion of medicaments or antimicrobial agents in the adhesive is also contemplated, as described in U.S. Pat. Nos. 4,310,509 and 4,323,557, the disclosures of which are incorporated herein by reference in their entirety.

Referring back to the drawings, the article 100 comprises a carrier comprising at least two spaced-apart sections 50. The carrier may be fabricated from a rigid material or a semirigid material. That is, the carrier is fabricated from a self-supporting material. Nonlimiting examples of suitable self-supporting materials include cellulosic sheet materials (e.g., self-supporting heavy-duty paper, cardstock, cardboard) and plastic materials (e.g., self-supporting polymeric sheet materials comprising polyethylene, polypropylene, polyester, polycarbonate, derivatives of any one of the foregoing, and mixtures of any two or more of the foregoing). In contrast to a carrier fabricated from a rigid, self-supporting material, a carrier fabricated from a semirigid material can permit an operator to flex or bend the backing 30 while the carrier section 50 is attached thereto (not shown). Advantageously, this permits the operator to flex or bend the carrier while applying the article 100 to a nonplanar (e.g., curved) surface (not shown).

In a preferred embodiment, the carrier may comprise a self-supporting paper of cardboard material that is coated with a polymer (e.g. a polyethylene/vinyl acetate copolymer).

The carrier sections 50 are releasably adhered to the first major surface 31 of the backing 30. Methods of releasably attaching a carrier to a polymeric film are described in U.S. Pat. No. 6,169,224, which is incorporated herein by reference in its entirety. One method described therein utilizes a low adhesion coating on the top face of a polymeric film to form a heat seal bond between the carrier and the film. Thus, in any embodiment, the backing 30 of an article 100 of the present disclosure optionally may comprise a low adhesion coating on all or a portion of its first major surface 31. Another means to releasably attach the carrier sections 50 to the first major surface 31 includes the use of a low-tack pressure-sensitive adhesive or by the use of a pattern that adheres less than 100% of the carrier to the backing.

Each of the carrier sections 50 comprises a central edge (52) and one or more peripheral edges (edges 53, 54, and 55, respectively). The central edges 52 extend from first portion “A” to a spaced-apart second portion “B” of the perimeter edge 34 of the backing 30. Together with the spaced-apart perimeter portions (A and B, respectively) of the backing 30, the central edges 52 substantially define an object-conformable region 37 of the backing 30. Flanking each side of the object-conformable region 37 are the anchor regions 38 of the backing 30.

FIG. 2c also shows a hypothetical midline 39 of the object-conformable region 37. The midline 39 extends from the first perimeter portion “A” to the second perimeter portion “B” of the object-conformable region 37. In the illustrated embodiment, the midline 39 defines a straight line. In any embodiment, the midline can define a substantially straight line. In use, at least a portion of the midline 39 of the object-conformable region 37 preferably is superimposed over the medical device (not shown) as the device is secured to a surface (not shown).

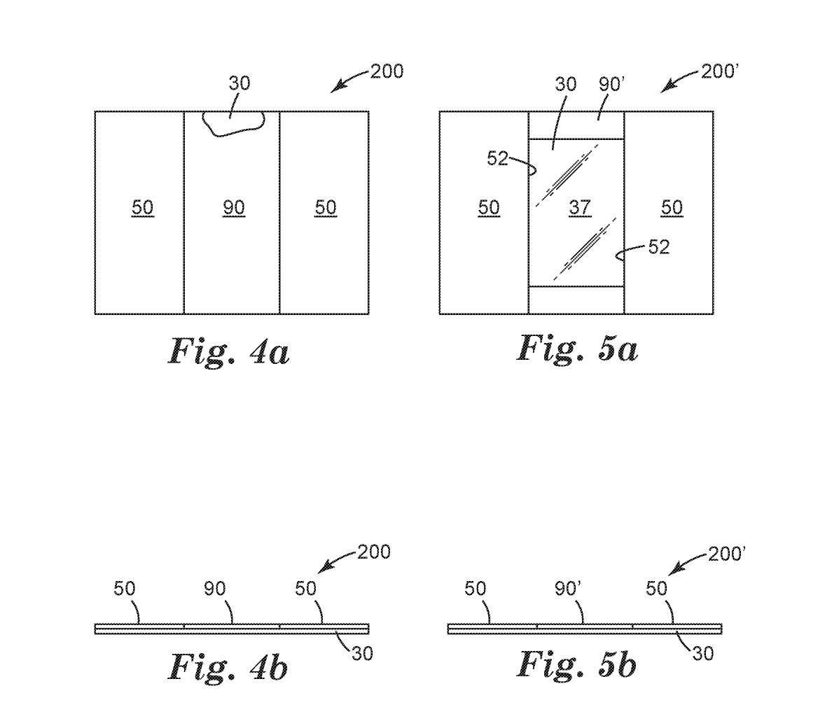

Optionally, each carrier section 50 further may comprise one or more carrier tabs 59. The carrier tabs 59 extend beyond the peripheral edge 34 of the backing 30 and can be used to facilitate detachment of the carrier from the backing 30. In a preferred embodiment, the carrier tabs 59 are positioned opposite the central edge 52 of the respective carrier section. Advantageously, this positioning permits the operator to grasp the carrier tab 59 and apply a force toward the central edge 52 when using the article to secure an object as Shown in FIG. 4a-d and described below. In addition, the tabs 59 can be used to separate the carrier sections 50 from the backing 30 after the backing has been applied to a surface.

In any embodiment, the second major surface 32 of the object-conformable region 37 of the article 100 may not include an adhesive coated thereon. Advantageously, this embodiment can provide easier release of the secured object (e.g. medical device 30) when removing the securement article. In addition, these embodiments can preclude the transfer of adhesive residue from the securement article to the medical device when the conforming securement article is removed. Without being bound by theory, in these embodiments not shown), movement of a secured medical device in a direction substantially parallel to the midline of the object-conformable region may be substantially inhibited by frictional forces between the materials comprising the external surface of the secured object (e.g., medical device) and the materials comprising the backing.

In any embodiment, the article 100 may comprise an indicium (e.g., a mark or a line, not shown) that demarks at least a portion (e.g., the midline 39) of the object-conformable region 37. Advantageously, the indicium can facilitate the proper positioning of the article 100, relative to the object to be secured and/or a relative to a location or landmark on the surface to which the object is to be secured, during the securement process. The indicium can be produced by a variety of methods known in the art such as, for example, printing a mark or a plurality of marks (e.g., a dotted line) onto the backing. Alternatively, or additionally, the indicium may comprise a notch in the backing (e.g., at the perimeter of the backing) or a perforation in the backing or a plurality of notches or perforations in the backing (not shown).

In any of the embodiments, the adhesive may be coated onto the second major surface of the backing using any suitable coating process known in the art (e.g., knife coating, gravure coating, kiss coating, die coating, spray coating). In any embodiment, the adhesive may be pattern-coated (not shown). In one embodiment, a portion of the adhesive may be pattern-coated as a strip running generally along the midline of the object-conformable region (not shown). In this embodiment, the adhesive strip may serve as an index mark to align the article with the medical device and may hold the medical device in a particular location or orientation relative to the securement article during the secural process.

Adhesives of the present disclosure may comprise silicone adhesives. Silicone-containing pressure-sensitive adhesives may be particularly preferred in applications where the article is adhered to skin and/or other surfaces. Examples of suitable silicone adhesives for use in articles of the present disclosure can be found in International Patent Publication No. WO 2010/056544, which is incorporated herein by reference in its entirety.

In addition, the article 100 may comprise an optional liner 60. The liner 60, when present, is releasably adhered to at least a portion of the adhesive layer 36 opposite the backing 30, thereby sandwiching at least a portion of the adhesive layer 36 between the backing 30 and the liner 60. In any embodiment, the area defined by the liner 60 may be at least coextensive with the area defined by the perimeter edge 34 of the backing 30, as shown in the illustrated embodiment of FIGS. 2a-c. In any embodiment, the liner 60 may further comprise one or more liner tab 62, which extends beyond the perimeter edge 34 of the backing. The tab 62 easily can be grasped by an operator and used to peel the liner 60 away from the adhesive layer 36 before or during the process of securing an object using the article 100. Optionally, the liner may comprise a plurality of liner sections (not shown). Each section of liner 60 may comprise a liner tab 62.

Liners which are suitable for use in the article of the present disclosure can be made of Kraft papers, polyethylene, polypropylene, polyester or composites of any of these materials. The liners are preferably coated with release agents such as fluorochemical or silicones. For example, U.S. Pat. No. 4,472,480, the disclosure of which is incorporated herein by reference in its entirety, describes low surface energy perfluorochemical liners. The preferred liners are papers, polyolefin films, or polyester films coated with silicone release materials. Examples of commercially available silicone coated release papers are POLYSLIK silicone release papers available from James River Co., H. P. Smith Division (Bedford Park, Ill.) and silicone release papers supplied by Daubert Chemical Co. (Dixon, Ill.). A preferred liner is 1-60BKG-157 paper liner available from Daubert, which is a super calendared Kraft paper with a water-based silicone release surface.

Other combinations of adhesives and liners are contemplated for use with embodiments according to the present invention. Those skilled in the art will be familiar with the processes of testing a new adhesive against different liners or a new liner against different adhesives to arrive at the combination of qualities desired in a final product. The considerations pertinent to the selection of a silicone release liner can be found in Chapter 18 of the Handbook of Pressure Sensitive Adhesive Technology, Van Nostrand Reinhold, 1982, pp. 384-403. U.S. Pat. No. 4,472,480 also describes considerations pertinent to the selection of a perfluoropolyether release liner.

Conformable securement articles of the present disclosure are used in a method of securing a medical device to a surface. In contrast to existing methods, the articles have features that enable the method to provide easier and more consistent application of the securement article to the medical device and surface, improved conformance of the securement article with the external surface features of the medical device, improved long-term securement of the medical device, and a reduced risk of accumulation of undesirable residue between the medical device and the surface.

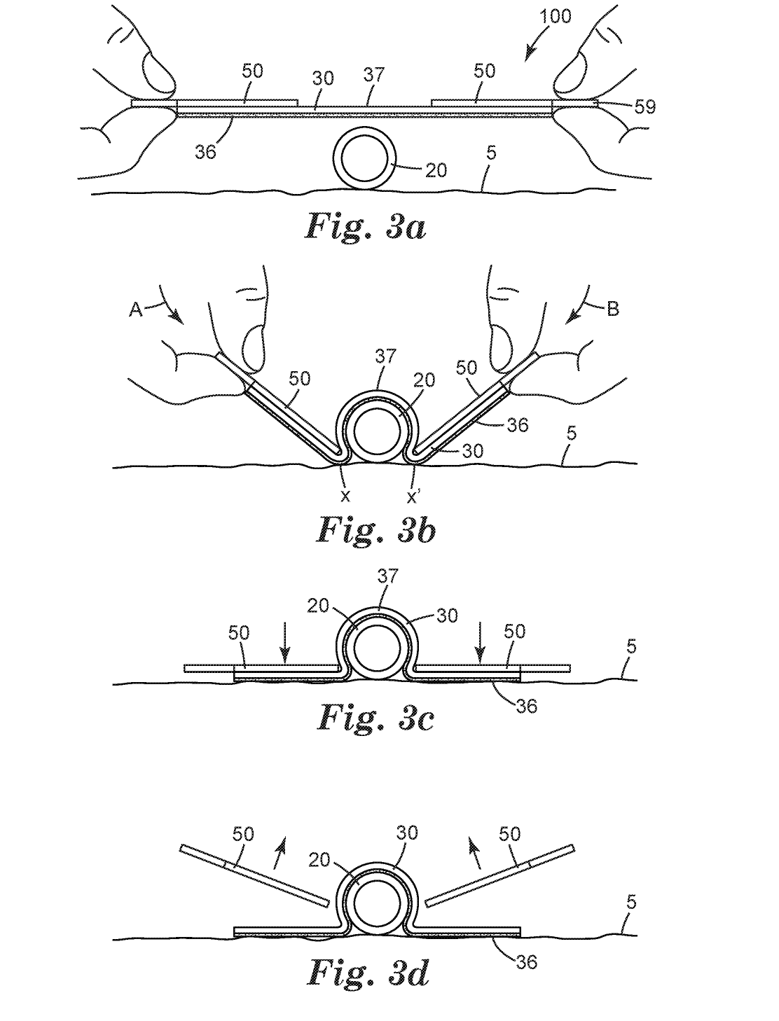

The present disclosure also provides a method of securing an object (e.g., a medical device) to a surface. FIG. 3a-d show side views of intermediate steps of one embodiment of a method of securing an object (e.g., a medical device) according to the present disclosure. Initially, the method comprises providing an object to be secured (e.g., medical device 20 such as a catheter, for example), a conformable securement article 100 according to the present disclosure, and a surface 5 (e.g., a patient's skin) on which to secure the device 20. The object to be secured has a topological shape.

The method further includes the step of positioning the device 20 and article 100 proximate the surface 5 to which the device is to be secured, as shown in FIG. 3a. Typically, a liner, if present on the article 100, is removed to expose the adhesive layer 36 before positioning the article proximate the device 20 and surface 5. While positioning the article 100, the article can be grasped by the carrier tabs 59, if present, as shown in FIG. 3a. Advantageously, this can help avoid contact between the adhesive layer 36 and the operator's hands. The article 100 is positioned with the object-conformable region 37 of the backing 30 proximate the device 20.

The method further includes using at least one section of the carrier to conform the backing to at least a portion of the topological shape. After positioning the article 100 and device 30, the operator uses each of the carrier sections 50 to urge (i.e., moving the respective carrier sections generally in the directions shown by the arrows “A” and “B”, respectively, in FIG. 3b) a portion of the backing 30 toward a location (X and X′, respectively) where the device 20 contacts the surface 5. In the process of moving the carrier sections 50, the object-conformable region 37 of the backing 30 substantially conforms to the topological shape of the object (device 20). “Conforms to the topological shape”, as used herein, means at least a portion of the backing assumes a shape that is substantially similar to a portion of the 3-dimensional shape of the object to be secured to a surface and, thereby, at least partially envelopes the object to be secured. In a preferred embodiment, conforming to the topological shape comprises substantially excluding trapped air between the backing, the object to be secured, and/or the surface to which the object is to be secured.

In some embodiments (not shown), each of the two carrier sections 50 can be moved sequentially (i.e., one carrier section is first used to conform a part of the securement region with the object to be secured and to contact one border of the securement region with the surface and then the other carrier section is used to conform another part of the securement region with the topological shape of the object to be secured and to contact another border of the securement region with the surface). In some embodiments, the carrier sections 50 can be moved simultaneously to conform the object-conformable region 37 to the topological shape of the object (device 20) to be secured and to contact spaced-apart borders of the securement region 37 with the surface 5.

The method further includes securing the backing 30 of the article 100 to the surface 5. This step may be performed with one carrier section 50 before (not shown) or after using the other carrier section to conform the object-conformable region 37 of the backing 30 to the object (e.g., device 20). In any embodiment, securing the article 100 to the surface 5 may comprise using the central edge 52 of at least one section 50 of the carrier to cause contact between the adhesive layer 36 and a portion of the surface 5 proximate the device 20. For example, the carrier sections 50 can be urged (e.g., manually) toward the surface 5, thereby placing any portion of the backing 30 that is not enveloping the device 20 proximate the surface, as shown in FIG. 3c. The adhesive layer 36 secures the backing 30 to the surface. In one embodiment (not shown), if the article comprises a semirigid carrier, one or both of the carrier sections may be flexed or bowed, either sequentially or simultaneously, and a rolling motion directed outward from the securement region can be used to bring the remainder of the backing into contact with the surface and arrive at the configuration shown in FIG. 3c.

Optionally, the method further comprises the step of removing one or more of the carrier sections 50 from the backing, as shown in FIG. 3d. Preferably, this step is performed after the backing 30 is secured to the surface 5. The carrier tabs 59, if present, can be grasped and pulled away from the surface 5 to release the carrier sections 50 from the backing 30.

It is contemplated that, in some embodiments of the method, one or more carrier sections may be left in place after securing the object. In these embodiments (not shown), the one or more carrier sections may provide structural support to resist movements of the backing that may otherwise cause premature separation of the adhesive layer from the surface.

In any embodiment, articles of the present disclosure may comprise one or more self-supporting scaffold members that are releasably adhered to same surface of the backing as the carrier sections. FIGS. 4a-b and 5a-b show two embodiments of an article (200 and 200′, respectively) comprising a scaffold member. The scaffold member superimposes at least a portion of the object-conformable region of the backing. The scaffold member may be fabricated using any material suitable for use as a carrier and can be releasably adhered to the backing using the same processes that are used to adhere the carrier to the backing. Preferably, the scaffold member is fabricated from the same material as the carrier and is physically separated from the carrier sections using a process such as controlled-depth die cutting, for example.

FIG. 4a-b show two views of one embodiment of a conforming securement article according to the present disclosure. The article 200 comprises a backing 30 that has two carrier sections 50 and one scaffold member 90 releasably adhered to one major surface and an adhesive layer 36 adhered to at least a portion of the other major surface. The central edges 52 of the carrier sections 50 define opposing borders of an object-conformable region 37 of the backing 30. The scaffold member 90 substantially superimposes the object-conformable region 37 of the backing 30. The scaffold member 90 can provide structural support to keep the carrier sections 50 spaced-apart until the article 200 is used to secure an object to a surface. For example, the scaffold member 90 can keep the carrier sections 50 spaced apart during or after the process of removing a liner (not shown), if present. In addition, in any embodiment, the scaffold member 90 can remain adhered to the backing 30 until the carrier sections are used to conform the backing to the topological shape of at least a part of the object to be secured. Accordingly, as the backing conforms to the object to be secured, a portion or all of a low-adhesion seal (not shown) between the scaffold member 90 and the backing is disrupted and the scaffold member 90 separates from the backing 30. Thus, an advantage of this embodiment is that the scaffold member can maintain the carrier sections in a spaced-apart configuration until the object to be secured is at least partially enveloped by the backing.

FIG. 5a-b show two views of an alternative embodiment of a conforming securement article according to the present disclosure. The article 200′ comprises a backing 30 that has two carrier sections 50 and two scaffold members 90′ releasably adhered to one major surface and an adhesive layer 36 adhered to at least a portion of the other major surface. The central edges 52 of the carrier sections 50 define opposing borders of an object-conformable region 37 of the backing 30. In this embodiment, the scaffold members 90′ extend from one central edge 52 to the other along the peripheral edge 34 of the backing 30, essentially framing, with the carrier sections 50 the central portion of the object-conformable region 37 of the backing 30. As discussed above, the scaffold members 90′ can provide structural support to keep the carrier sections 50 spaced-apart until the article 200′ is used to secure an object to a surface. Advantageously, because the central portion of the object-conformable region is not completely superimposed by the scaffold members 90′, the object to be secured (not shown), can be viewed through the backing 30, if the article 200′ includes an optically transmissive (e.g., transparent or translucent) backing 30.

As discussed above, scaffold members provide temporary structural support for the object-conformable region of the backing. Although the scaffold members (90 and 90′, respectively) of the illustrated embodiments extend from one central edge 52 to the other, that particular feature is not a requirement. Other useful shapes and configurations of scaffold members will be apparent to a person having ordinary skill in the art.

In any embodiment, the scaffold members (e.g., scaffold members 90 and 90′ discussed above) and/or carrier sections (50, discussed above) may be fabricated from an optically transmissible (e.g., translucent or transparent) material such as a clear polymer film, for example. Advantageously, this can provide the operator with a better view of the object to be secured and the surface to which the object will be secured during the process of using the article according to the methods described herein.

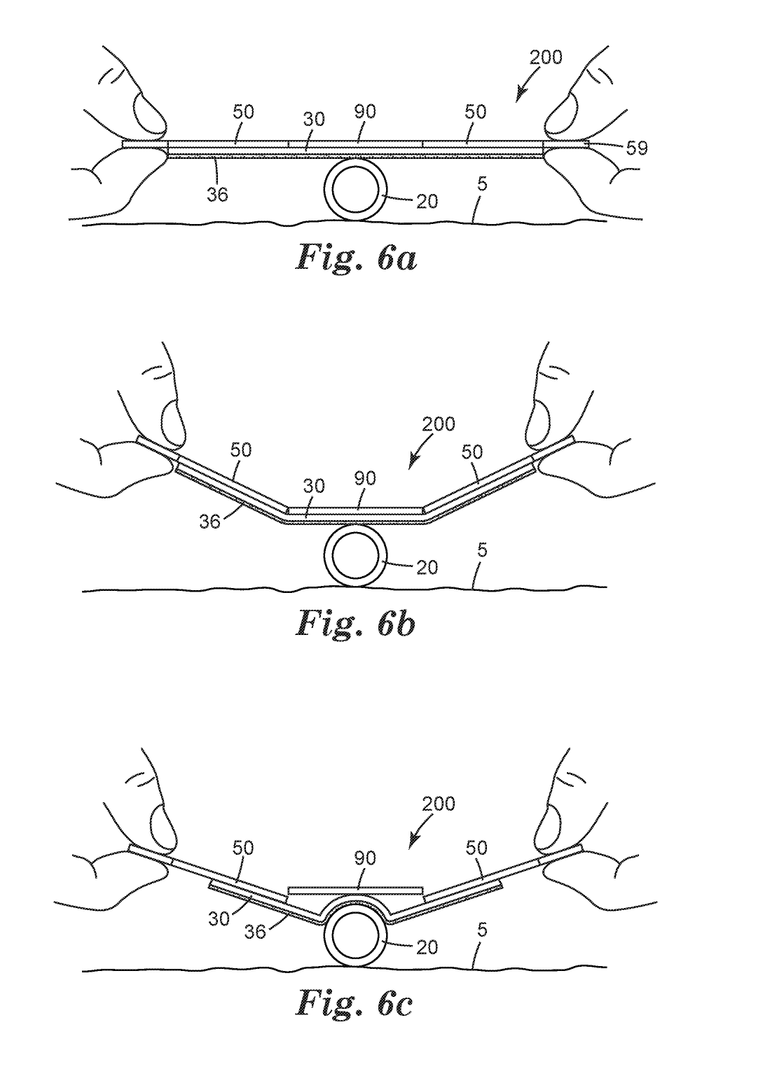

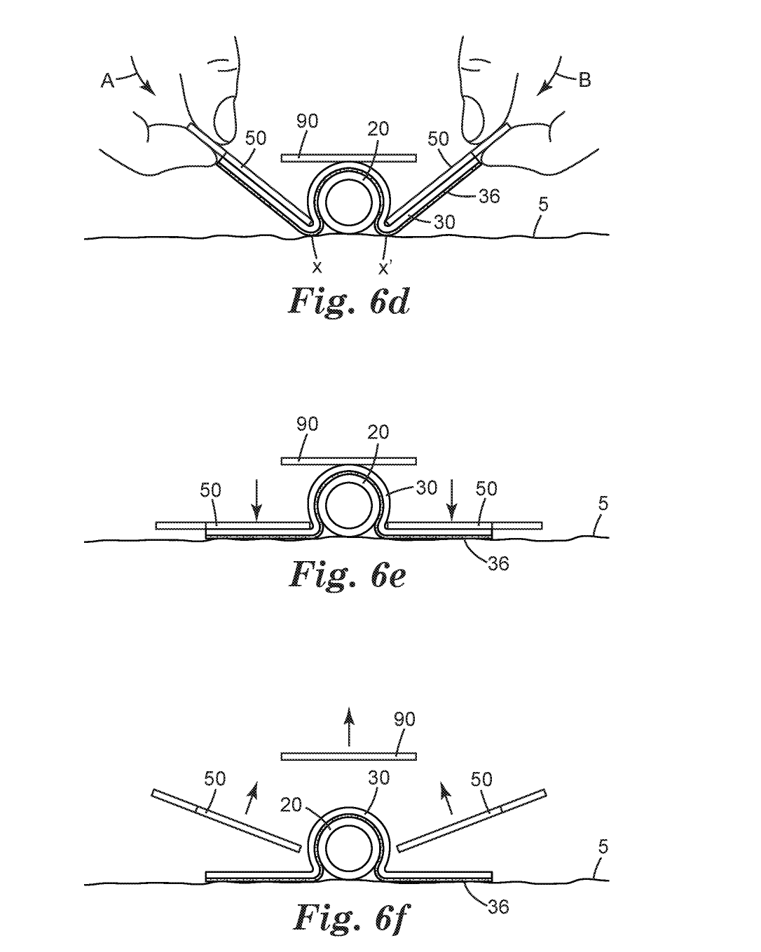

FIGS. 6a-f show side views of several intermediate steps of one embodiment of a method of securing an object (e.g., a medical device such as a catheter, for example) using the conformable securement article of FIGS. 4a-b. The method includes the step of positioning the device 20 and article 200 proximate the surface 5 to which the device is to be secured, as shown in FIG. 3a. Typically, a liner (not shown), if present on the article 200, is removed to expose the adhesive layer 36 before positioning the article proximate the device 20 and surface 5. While positioning the article 200, the article can be grasped by the carrier tabs 59, if present. The article 200 is positioned with the object-conformable region 37 of the backing 30 and scaffold member proximate the device 20, as shown in FIG. 6a. Optionally, one or both of the carrier sections 50 can be inclined away from the surface 5, as shown in FIG. 6b. Inclining the carrier sections 50 as shown advantageously can facilitate (e.g., by slightly stretching the backing 30) detachment of the scaffold member 90 from the backing 30 during subsequent steps.

The method further includes using at least one section of the carrier to conform the backing to at least a portion of the topological shape. This may be accomplished by urging the carrier sections 50 toward the surface 5 by moving the article-grasping means (e.g., hands) that are grasping the carrier sections 50 generally in a direction toward the surface 5, as shown by the arrows in FIG. 6c. This motion causes the backing 30 to begin conforming to the topological shape of the device 20 and causes the scaffold member 90 to continue detaching from the backing 30. The carrier sections 50 may be moved sequentially or they may be moved simultaneously during this step. The carrier sections 50 are used to direct portions of the backing 30 toward a location (X and X′, respectively, shown in FIG. 6d) where the device 20 contacts the surface 5. In the process of moving the carrier sections 50 to these locations, the object-conformable region 37 of the backing 30 substantially conforms to the topological shape of the object (device 20) and the scaffold member 90 is substantially detached from the backing 30.

The method further includes securing the backing 30 of the article 200 to the surface 5. This step may be performed with one carrier section 50 before (not shown) or after using the other carrier section to conform the object-conformable region 37 of the backing 30 to the object (e.g., device 20). In any embodiment, securing the article 200 to the surface 5 may comprise using the central edge 52 of at least one section 50 of the carrier to cause contact between the adhesive layer 36 and a portion of the surface 5 proximate the device 20. For example, the carrier sections 50 can be urged (e.g., manually) toward the surface 5, thereby placing any portion of the backing 30 that is not enveloping the device 20 proximate the surface, as shown in FIG. 6e. The adhesive layer 36 secures the backing 30 to the surface. In one embodiment (not shown), if the article comprises a semirigid carrier, one or both of the carrier sections may be flexed or bowed, either sequentially or simultaneously, and a rolling motion directed outward from the securement region can be used to bring the remainder of the backing into contact with the surface and arrive at the configuration shown in FIG. 6e.

Optionally, the method further comprises the step of removing one or more of the carrier sections 50 and/or scaffold member 90 from the backing 30, as shown in FIG. 6f Preferably, this step is performed after the backing 30 is secured to the surface 5. The carrier tabs 59, if present, can be grasped and pulled away from the surface 5 to release the carrier sections 50 from the backing 30. Any free edge of the scaffold member 90 can be grasped for removal of the scaffold member.

It is contemplated that, in some embodiments of the method, one or more carrier sections may be left in place after securing the object. In these embodiments (not shown), the one or more carrier sections may provide structural support to resist movements of the backing that may otherwise cause premature separation of the adhesive layer from the surface.

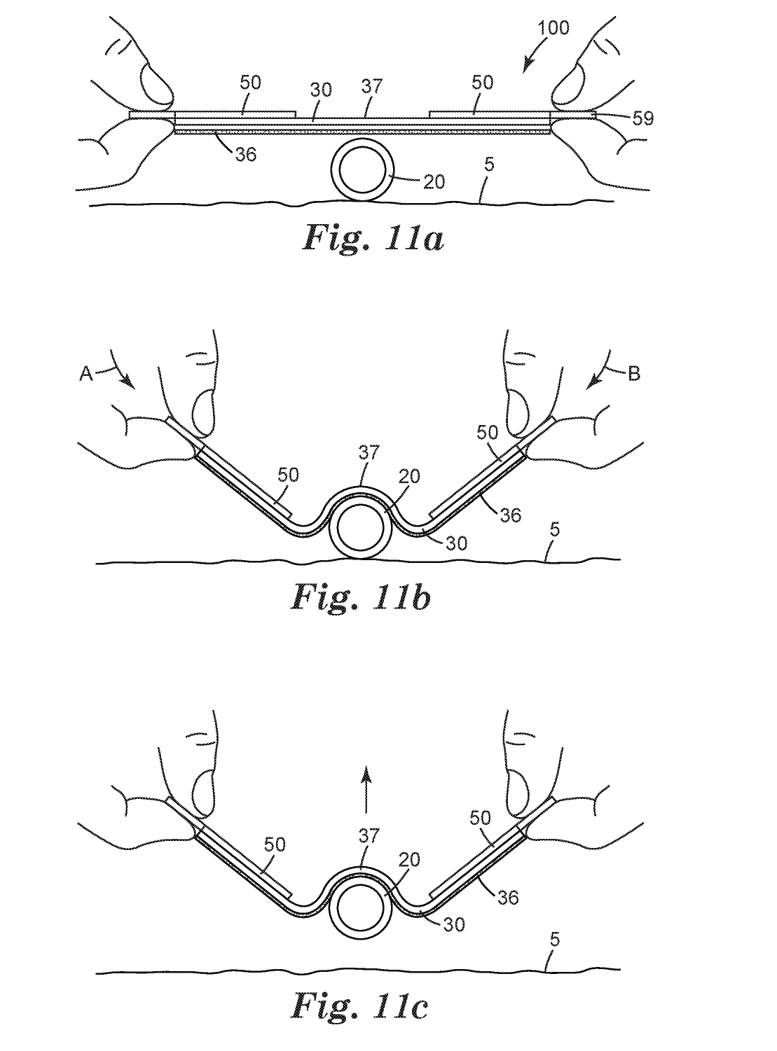

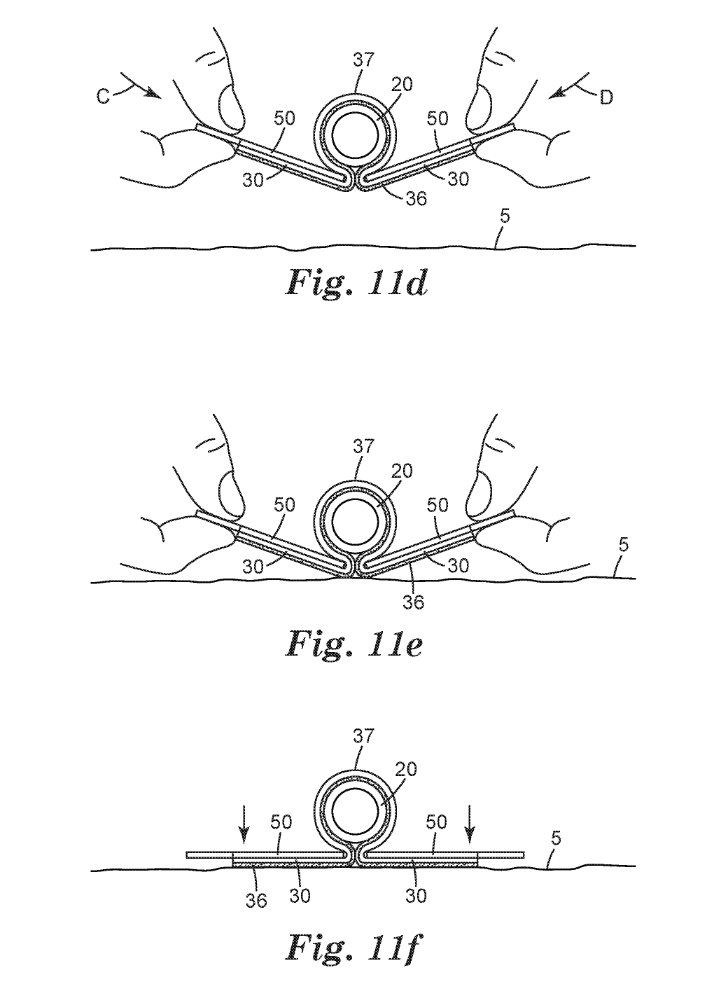

Articles of the present disclosure further may be used in a method of securing an object to a surface such that the object is held off the surface by the article. FIGS. 11a-f show side views of several intermediate steps of one embodiment of a method of securing an object (e.g., a medical device such as a catheter, for example) to a surface, such that the object is held off the surface by the article, using the conformable securement article 100 of FIGS. 2a-c. The method can be used with any securement article described herein.

The method includes the step of positioning the object (e.g., device 20) and article 100 proximate the surface 5 to which the device is to be secured, as shown in FIG. 11a. Typically, a liner (not shown), if present on the article 100, is removed to expose the adhesive layer 36 before positioning the article proximate the device 20 and surface 5. While positioning the article 100 relative to the object to be secured and the surface 5, the article 100 can be grasped by carrier tabs 59, if present. The article 100 is positioned with the object-conformable region 37 of the backing 30 proximate the device 20, as shown in FIG. 11a. The method further includes using at least one section 50 of the carrier to conform the backing to at least a portion of the topological shape of the object (device 20). After positioning the article 100 and device 30, the operator can use each of the carrier sections 50 to urge (i.e., moving the respective carrier sections generally in the directions shown by the arrows “A” and “B”, respectively, in FIG. 11b) a portion of the backing 30 around a portion of the circumference of the device 20. In the process of moving the carrier sections 50, the object-conformable region 37 of the backing 30 substantially conforms to the topological shape of the object (device 20) as shown in FIG. 11b.

At this point, the object to be secured (device 20) temporarily can be lifted off the surface 5 (in the direction of the arrow, as shown in FIG. 11c) in order to make at least a portion of the entire circumference of the device 20 accessible to the conformable backing 30. The device 20 remains adhered to the article 100 via the adhesive layer 36. The carrier sections 50 can then be used to wrap the entire circumference of the device 20 with the backing 30, essentially bringing the adhesive layer 36 on the backing 30 into contact with itself after fully engulfing the device 20. This can be accomplished by urging the carrier sections 50 toward each other using motions designated by arrows “C” and “D” in FIG. 11d. Subsequently, the object (device 20) that is engulfed by the article 100 can be positioned at the proper location on the surface 5, as shown in FIG. 11e, and the remainder of the backing 30 can be brought into contact with the surface 5, as shown by the arrows in FIG. 11f. The carrier sections 50 then can be separated from the backing 30, as shown in FIG. 3d. Advantageously, this method minimizes or eliminates contact between the object and the surface which, in certain clinical applications, may result in significantly reduced incidence of skin irritation and/or infection.

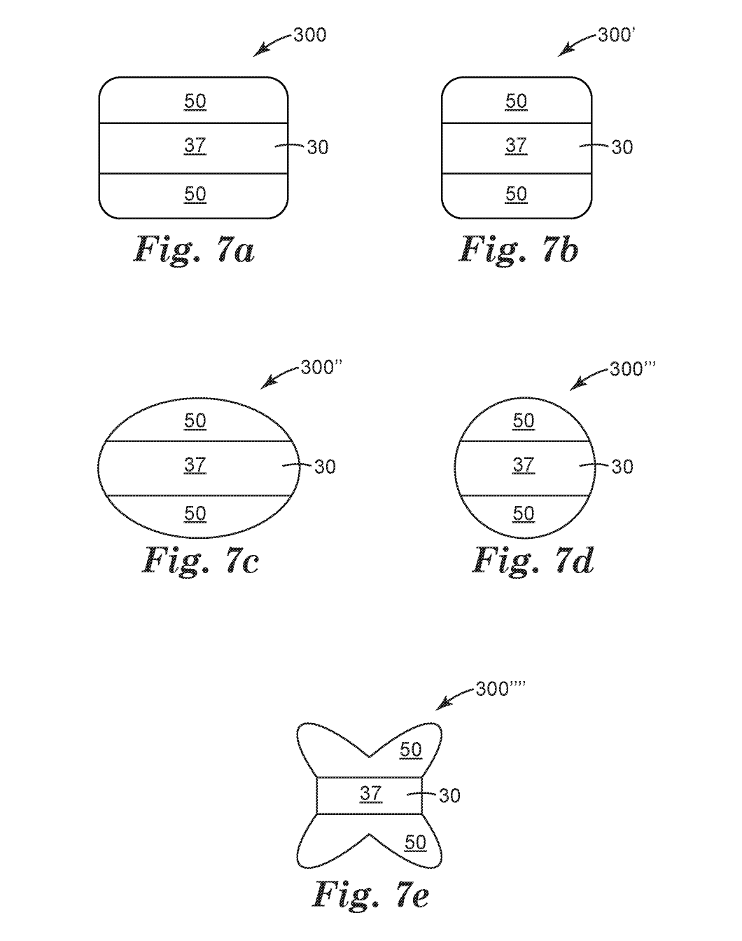

Articles of the present disclosure include a variety of two-dimensional shapes and sizes, which may be defined by the perimeter edge of the backing and can be selected with respect to the shape and size of the article to be secured to a surface and/or the shape and size of the particular surface to which the article is to be secured. Accordingly, the articles may take the shape of a rectangle, square, oval, circle, or star, for example. FIGS. 7a-e show plan views of variously-shaped articles (300, 300′, 300″, 300′″, and 300′″, respectively) according to the present disclosure. Similar to the construction shown in FIG. 2c, the articles shown in FIGS. 7a-e include two carrier sections 50 located on opposite sides of an object-conformable region 37 of the backing 30.

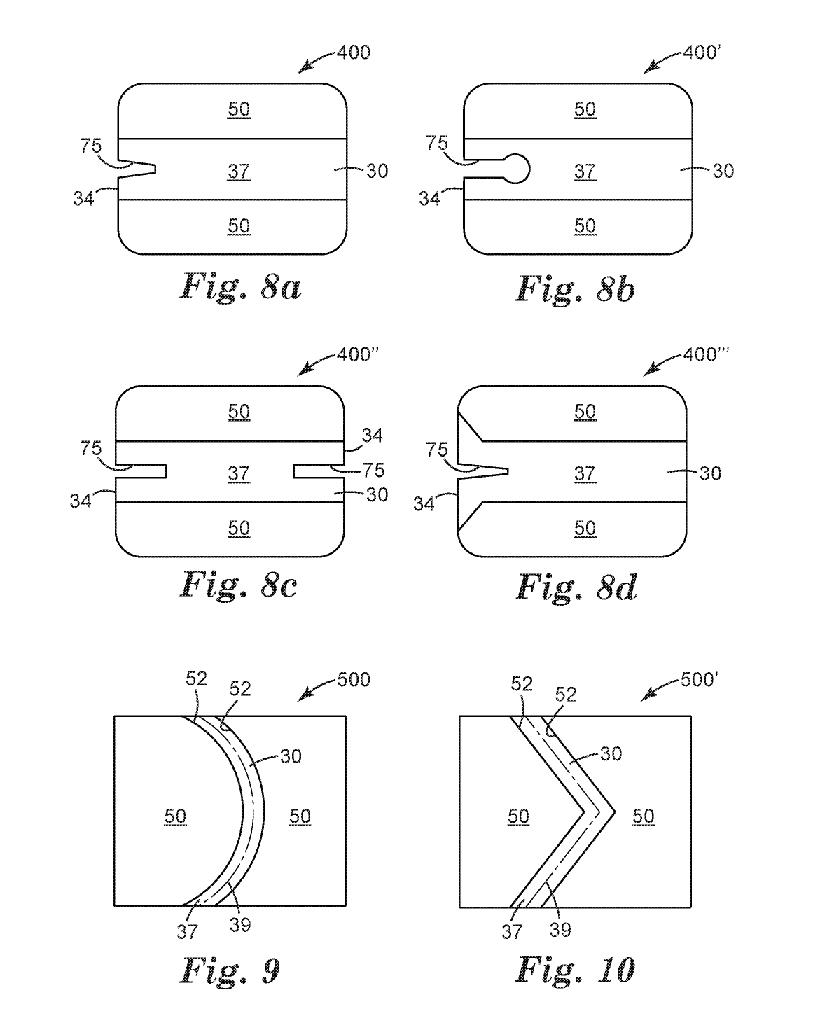

Articles of the present disclosure can be adapted to exclude a particular feature of the object to be secured to a surface from being enveloped by the object-conformable region of the backing. An example of such an adaptation is providing a slit in the peripheral edge of the backing in the object-conformable region. This adaptation may be particularly useful when securing objects such as a medical device than includes a portion (e.g., a catheter hub, an EKG or EMG electrode) that may require intermittent access and/or may require limited mobility with respect to the rest of the secured object (e.g., the catheter line, the electrode lead). FIGS. 8a-d show plan views of four exemplary embodiments of articles (400, 400′, 400″, and 400′″, respectively) with a slit 75 in the peripheral edge 34 of the object-conformable region 37 of the backing 30. The slit 75 may be configured in any one of a variety of shapes and sizes, as illustrated in FIGS. 8a-d. In addition, it is contemplated that the article may comprise a plurality of slits 75, as illustrated in the exemplary embodiment of FIG. 8c. Also shown in FIGS. 8a-d are the carrier sections 50 of each of the respective articles.

In addition, it is contemplated that the shape and/or size of the object-conformable region of the backing of articles according to the present disclosure can be selected according to the shape and/or size of the topological shape of the object to be secured. FIGS. 9 and 10 show plan views of two examples of articles (500 and 500′, respectively) having a shaped object-conformable region. The article 500 in FIG. 9 comprises two carrier sections (sections 50 and 50, respectively) having central edges (52 and 52, respectively) that define a curvilinear object-conformable region 37 of the backing 30. The central edges 52 and 52 are complementary-shaped. In any embodiment of an article according to the present disclosure, the central edges that define borders of an object-conformable region can be complementary-shaped. Also shown in FIG. 9 is the hypothetical midline 39 of the object-conformable region 37. In this embodiment, the midline 39 defines a curved line.

The article 500′ in FIG. 10 comprises two carrier sections (sections 50 and 50, respectively) having central edges (52 and 52, respectively) that define an angular object-conformable region 37 of the backing 30. The central edges 52 and 52 are complementary-shaped. Also shown in FIG. 10 is the hypothetical midline 39 of the object-conformable region 37. In this embodiment, the midline 39 defines an angular line.

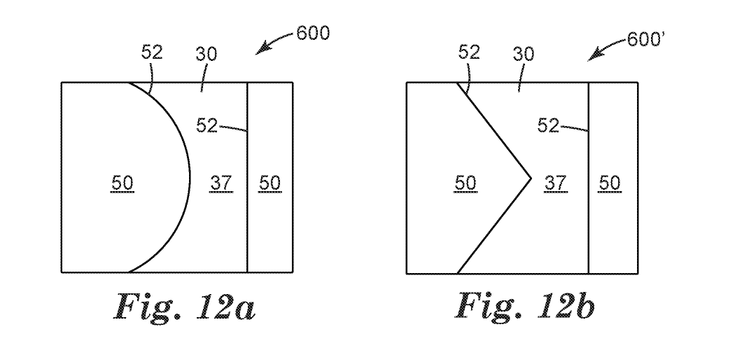

Articles with central edges 52 that are complementary-shaped, as shown in FIGS. 9 and 10 may be desired in certain applications (e.g., when the complementary-shaped central edges 52 substantially conform to the shape of the article to be secured to a surface). However, complementary-shaped central edges 52 are not required. In any embodiment, the central edges 52 of the carrier sections 50 may be non-complementary, as shown in FIGS. 12a-b. In the illustrated embodiments, the non-complementary central edges 52 are configured such that the articles 600 and 600′ can be used to secure an article with one side that is substantially liner and another side that is substantially curved (FIG. 12a) or angular (FIG. 12b). A person having ordinary skill in the art will recognize that the central edges of the carrier sections can be shaped and dimensioned to conform to a variety of asymmetrically-shaped objects.

### EMBODIMENTS

Embodiment 1 is an article, comprising:

a conformable backing having top and bottom major surfaces and a perimeter edge;

an adhesive coated on at least a portion of the bottom major surface of the backing; and

a self-supporting carrier releasably adhered to the top major surface of the backing, the carrier having spaced-apart first and second sections, each section having a central edge;

wherein the central edges define opposing borders of an object-conformable region of the backing.

Embodiment 2 is the article of embodiment 1, wherein the backing further comprises two conformable anchor regions located on opposite sides of the object-conformable region.

Embodiment 3 is the article of embodiment 1 or embodiment 2, wherein the backing comprises a sheet material comprising polymer film, a foam, a woven fabric, a nonwoven fabric, or a combination of any two or more of the foregoing sheet materials.

Embodiment 4 is the article of any one of the preceding embodiments, wherein the backing consists essentially of a polymer film.

Embodiment 5 is the article of embodiment 3 or embodiment 4, wherein the sheet material comprises an elastic sheet material.

Embodiment 6 is the article of any one of the preceding embodiments, wherein the central edges are substantially complementary shaped.

Embodiment 7 is the article of any one of embodiments 1 through 5, wherein the central edges are substantially non-complementary shaped.

Embodiment 8 is the article of any one of the preceding embodiments, wherein the object-conformable region is substantially transparent.

Embodiment 9 is the article of any one of the preceding embodiments, wherein the adhesive comprises an iso-octyl acrylate:acrylamide copolymer, an isooctyl acrylate:ethyleneoxide acrylate:acrylic acid terpolymer, a derivative of either of the foregoing adhesives, a silicone adhesive, or a mixture of any two or more of the foregoing adhesives.

Embodiment 10 is the article of any one of the preceding embodiments, wherein the adhesive is pattern-coated.

Embodiment 11 is the article of any one of the preceding embodiments, further comprising a liner releasably adhered to the adhesive.

Embodiment 12 is the article of any one of the preceding embodiments, wherein the carrier comprises a material selected from the group consisting of self-supporting heavy-duty paper, cardstock, cardboard, and self-supporting polymeric sheet materials, and a mixture or a combination of any two or more of the foregoing materials.

Embodiment 13 is the article of any one of the preceding embodiments, wherein the carrier comprises a material that is optically-transmissive.

Embodiment 14 is the article of any one of the preceding embodiments, wherein the object-conformable region comprises a first midline, wherein the first midline defines a substantially straight line.

Embodiment 15 is the article of any one of embodiments 1 through 12, wherein the object-conformable region comprises a first midline, wherein the first midline defines a curved line.

Embodiment 16 is the article of any one of embodiments 1 through 12, wherein the object-conformable region comprises a first midline, wherein the first midline defines an angular line.

Embodiment 17 is the article of any one of the preceding embodiments, further comprising a third section of the carrier releasably adhered to the top face of the object-conformable region of the backing.

Embodiment 18 is the article of any one of the preceding embodiments, wherein the object-conformable region further comprises at least one slit located along the perimeter of the backing.

Embodiment 19 is the article of embodiment 18, wherein the region comprises a plurality of slits, at least one slit located along the perimeter of the backing at one end of the object-conformable region and another slit located along the perimeter of the backing at the other end of the object-conformable region.

Embodiment 20 is the article of any one of the preceding embodiments, wherein the carrier further comprises a tab.

Embodiment 21 is the article of embodiment 20, wherein the tab is positioned along a peripheral edge of at least one section of the carrier, wherein the peripheral edge of the at least one section is opposite the central edge of the at least one section.

Embodiment 22 is the article of any one of the preceding embodiments, further comprising an adhesive strip releasably adhered to the top face of the carrier.

Embodiment 23 is the article of any one of the preceding embodiments, further comprising a reinforcement layer.

Embodiment 24 is the article of embodiment 23, wherein the reinforcement layer comprises a spunlace nonwoven fabric.

Embodiment 25 is the article of any one of the preceding embodiments, wherein the backing further comprises an indicium.

Embodiment 26 is the article of embodiment 25, wherein the indicium comprises a mark, a notch, or a perforation.

Embodiment 27 is the article of embodiment 25 or embodiment 26, wherein the indicium indicates a location proximate a midline of the object-conformable region.

Embodiment 28 is a method for securing an object to a surface, comprising:

providing an object having a topological shape, an article according to any one of the preceding embodiments, and a surface on which to secure the device;

positioning the device and article proximate the surface;

using at least one section of the carrier to conform the backing to at least a portion of the topological shape; and

securing the backing to the surface.

Embodiment 29 is the method of embodiment 28, wherein securing the backing to the surface comprises using the central edge of at least one section of the carrier to cause contact between the adhesive and a portion of the surface proximate the device.

Embodiment 30 is the method of embodiment 28 or embodiment 29, wherein using at least one section of the carrier comprises using both the first and second sections of the carrier.

Embodiment 31 is the method of embodiment 30, wherein using the first and second sections of the carrier further comprises sequentially using the first and second sections.

Embodiment 32 is the method of embodiment 30, wherein using the first and second sections of the carrier further comprises simultaneously using the first and second sections.

Embodiment 33 is the method of any one of embodiments 28 through 32, wherein using the central edge of at least one section comprises using the central edges of the first and second sections of the carrier.

Embodiment 34 is the method of embodiment 33, wherein using the central edge of the first and second sections of the carrier further comprises sequentially using the central edges of the first and second sections of the carrier.

Embodiment 35 is the method of embodiment 33, wherein using the central edge of the first and second sections of the carrier further comprises simultaneously using the central edges of the first and second sections of the carrier.

Embodiment 36 is the method of any one of embodiments 28 through 35, wherein using the central edge comprises using the central edge to reduce tenting between the backing and the surface.

Embodiment 37 is the method of any one of embodiments 29 through embodiment 36, wherein using the central edges comprises using the edges to provide contact between the article and the device substantially completely around a circumference of the device.

Embodiment 38 is the method of any one of embodiments 28 through 37, wherein the object comprises a medical device.

Embodiment 39 is the method of embodiment 38, wherein the medical device is selected from the group consisting of a tube, a catheter, or an electrode lead.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

### Example 1

### Construction of a Conformable Securement Article

A conformable securement article was constructed by adding a carrier as described herein to a transparent film dressing. A 3M™ TEGADERM transparent film dressing (part no. 1628) was obtained from 3M Company (St. Paul, Minn.). Two 2.7 cm×7 cm pieces of cardboard, having a thickness of 0.35 mm and a weight of 0.0187 g/cm², were laminated to the adhesive-coated side of two identically shaped pieces of paper that were fully-coated on one side with a repositionable Post-it adhesive similar to the 3M™ Repositionable Spray Adhesive Part No. 75 available from 3M Company (St. Paul, Minn.). Transfer tape (Part No. 924 transfer tape, available from 3M Company) was adhered to the paper on the side of the paper opposite the repositionable adhesive side. The cardboard was then pressed against the transfer tape to create a laminate with the cardboard on one side and the repositionable adhesive on the other side.

A 3M™ TEGADERM dressing was removed from its packaging and the carrier web was removed to expose the polyurethane film backing. The dressing was placed on a flat surface with the exposed backing side up. The two previously-made cardboard laminates were placed on the polyurethane film with the repositionable adhesive side down, such that the two laminates were side by side and parallel to each other, and had a gap of 1.2 cm between them. Subsequently, the excess polyurethane film and liner the dressing were cut along the border of the two cardboard laminates such that an approximately 7×7 cm conforming securement article similar to the one shown in FIG. 6b was obtained.

### Example 2

### Use of a Conformable Securement Article to Secure an Object to a Surface

The securement article of Example 1 was used to attach a piece of Foley catheter tube to a piece of cardboard using the method depicted in FIGS. 4a-d and described above. After removing the carrier sections, no tenting was observed between the polyurethane film, the tube, and the cardboard surface.

### Comparative Example 1

### Use of a Transparent Film Dressing to Secure an Object to a Surface

A 3M™ TEGADERM transparent film dressing (part no. 1685) was obtained from 3M Company. The dressing was used to attach a piece of Foley catheter tube to a piece of cardboard using the method depicted in FIGS. 1a-c and described above. Significant tenting was observed between the polyurethane film, the tube, and the cardboard surface.

### Example 3

### Construction of a Conformable Securement Article

3M DURAPORE tape (Part No. 1538-3) was obtained from 3M Company. The adhesive side of a strip of the tape was adhered to a siliconized paper liner while the conformable securement article was constructed. Two pieces of cardboard laminate having a repositionable adhesive on one side (as described in Example 1) were cut to match the 3-inch (7.5 cm) width of the tape. The cardboard laminates were adhered to the nonadhesive side of the tape with a gap of about 1.2 cm between the cardboard laminates.

### Example 4

### Use of a Conformable Securement Article to Secure an Object to a Surface

The securement article of Example 3 was used to attach a piece of Foley catheter tube to a piece of cardboard using the method depicted in FIGS. 4a-d and described above. After removing the carrier sections, no tenting was observed between the polyurethane film, the tube, and the cardboard surface.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A method for securing an object to a surface, comprising:
providing an object having a topological shape, an article according to any one of the preceding claims, and a surface on which to secure the device;
positioning the device and article proximate the surface;
using at least one section of the carrier to conform the backing to at least a portion of the topological shape; and
securing the backing to the surface.

2. The method of claim 2, wherein securing the backing to the surface comprises using the central edge of at least one section of the carrier to cause contact between the adhesive and a portion of the surface proximate the device.

3. The method of claim 1, wherein using at least one section of the carrier comprises using both the first and second sections of the carrier.

4. The method of claim 3, wherein using the first and second sections of the carrier further comprises sequentially using the first and second sections.

5. The method of claim 3, wherein using the first and second sections of the carrier further comprises simultaneously using the first and second sections.

6. The method of claim 1, wherein using the central edge of at least one section comprises using the central edges of the first and second sections of the carrier.

7. The method of claim 6, wherein using the central edge of the first and second sections of the carrier further comprises sequentially using the central edges of the first and second sections of the carrier.

8. The method of claim 6, wherein using the central edge of the first and second sections of the carrier further comprises simultaneously using the central edges of the first and second sections of the carrier.

9. The method of claim 6, wherein using the central edge comprises using the central edge to reduce tenting between the backing and the surface.

10. The method of claim 6, wherein using the central edges comprises using the edges to provide contact between the article and the device substantially completely around a circumference of the device.

11. The method of claim 1, wherein the object comprises a medical device.

12. The method of claim 11, wherein the medical device is selected from the group consisting of a tube, a catheter, or an electrode lead.

13. The method of claim 2, wherein the central edge extends from a first peripheral edge to a second peripheral edge.

14. The method of claim 6, wherein the central edge of the first section is spaced-apart from the central edge of the second section.
